(19) United States
(12) Patent Application Publication
(10) Pub. No.: US 2010/0305542 A1
(43) Pub. Date: Dec. 2, 2010

(54) Diaper or Diaper Like Undergarment and Separation Device for Undergarments

(76) Inventor: Gordon A. Stemple, Fresno, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 12/564,914

(22) Filed: Sep. 22, 2009

Related U.S. Application Data
(60) Provisional application No. 61/182,015, filed on May 28, 2009.

(51) Int. Cl. A61F 13/51

(52) U.S. Cl. 604/385.101

(57) ABSTRACT

An improved undergarment and a separation device for use with undergarments that are worn by persons afflicted with dual incontinence to separate urine and feces that collect in the undergarment. In one embodiment, the undergarment is provided with the separation device at or near the middle area of the inner layer of the undergarment to divide the inner layer into a front section and a back section. The separation device has a center divider that prevents migration and mixing of urine and feces. The upper surface of the center divider interconnects the undergarment and the wearer's body. The separation device can include a pair of wing members extending outward from the center divider that are configured to bend downward for positioning and to flex out against the wearer's legs to assist in holding the center divider in place. The separation device can also be utilized to retrofit existing undergarments.

### CROSS-REFERENCE TO RELATED APPLICATIONS

This patent application claims the benefit of U.S. Provisional Patent Application No. 61/182,015 filed May 28, 2009.

### STATEMENT REGARDING FEDERALLY SPONSORED RESEARCH

Not Applicable.

### BACKGROUND OF THE INVENTION

A. Field of the Invention

The field of the present invention relates generally to diaper or diaper like undergarments that are worn by persons unable or unwilling to control their bodily functions. In particular, the present invention relates to undergarments and devices therefor that are configured to keep urine and feces separate in a manner which substantially protects the wearer from diseases and the like. Even more particularly, the present invention relates to such undergarments which can be worn by adults.

B. Background

As the world's population ages, especially in the more advanced countries, people are generally living longer. As a consequence of people living into their late 70's, 80's or beyond, incontinence is becoming a greater problem than it has been in the past. Presently, the typical answer to the problem of how to handle persons having incontinence is to utilize a diaper like product that is configured for adults, such as Depends® and the like. Although these diaper and diaper like products generally perform well with regard to containing the person's bodily wastes, people who have incontinence suffer the indignity of having to sit in a mixture of their own feces and urine, sometimes for an extended period of time. While this is particularly true for older people while traveling on long trips, it is also typical for incontinent persons residing in hospitals, convalescent homes and other care locations, including persons in a home care arrangement. From a health standpoint the consequence of being subjected to a mixture of feces and urine for an extended period of time can be tragic, especially for elderly women who are highly subject to a urinary tract infections (hereinafter, “UTI”).

When the feces of elderly women come in contact with their vagina, the combination frequently causes a UTI, which in turn can cause them to become emotionally unstable and appear to have symptoms of dementia and Alzheimer's disease. Some doctors miss the causal connection between the UTI and these symptoms and the person then starts being treated for an illness they do not have. Even when the illness is properly detected, both the UTI and any emotional instability caused by the UTI can only be properly treated or cured by applying stronger and stronger types and doses of antibiotics. Unfortunately, this is an escalating event. As more frequent and stronger antibiotics are used to treat the UTI, the person often becomes antibiotic resistant. This phenomena leads to other illness, such as methicillin-resistant Staphylococcus aureus, MRSA, Clostridium difficile (also known as C. diff) and other nosocomial infections, which are infections that are a result of treatment in a hospital or other health care unit, such as a nursing home or the like. As well known in the art, certain nosocomial infections are already at epidemic proportions throughout the world. These nosocomial diseases are serious medical conditions and can be fatal if not treated soon enough or properly. Because touching contaminated people or physical objects that a contaminated person has touched can cause another person to contract these diseases, the diseases have crossed over to infect all age groups of the general population.

The cost of treating UTIs facing the world's elderly population and the nosocomial infections that are, at least in part, related to UTIs is in the $100's of billions per year range. Left uncorrected, these yearly costs could soar to the trillions of dollars.

Part of the reason for the prevalence of UTIs and various nosocomial infections in people who have incontinence is that, from a practical standpoint, health care workers generally find it easier to let the person discharge their bodily wastes into a diaper or diaper like product and then wash them later instead of helping the person get to a toilet before they discharge the wastes (which timing is not usually very predictable). Because the hospital or other health care unit generally houses a relatively large number of persons who are sick and/or have weakened immune systems, the opportunity for nosocomial infections is very high. In fact, the staff of the health care unit can, even when being very careful, spread diseases from one person to another.

A less drastic result of allowing urine and feces to mix in the same adult diaper that is applicable to both men and women, is the creation of a toxic mixture which is known to be irritating to the skin, through both surface irritation and dermal absorption. This also leads to external and internal systemic infections, which generally contributes to the global problems resulting from the overuse of antibiotics. The various diseases associated with non-UTI infections resulting from exposure to the mixture of feces and urine also contribute to nosocomial infections.

What is needed, therefore, is an improved diaper-like undergarment and a device for diaper-like undergarments that can be worn by the elderly or other persons who are experiencing incontinence problems to eliminate or at least substantially reduce the mixing of feces and urine and, as a result, the exposure of the person to such mixture. The improved diaper and device should be able to reduce the likelihood of a UTI and other diseases or health issues which are known to result from presently configured diapers and diaper-like undergarments. The diaper-like undergarment and device for undergarments should be configured to be worn by both children and adults. The device should be configured to be utilized with different types of undergarments.

### SUMMARY OF THE INVENTION

The improved diaper-like undergarment and the separation device for diaper-like undergarments of the present invention provides the benefits and solves the problems identified above. That is to say, the present invention discloses an improved diaper or diaper-like undergarment and separation device for undergarments which can be worn by persons having incontinence and similar problems that are configured to reduce the likelihood of UTIs, rashes and other diseases or health issues. The improved diaper or diaper-like undergarment of the present invention has front and rear sections that are separated by a barrier which maintains the urine in the front section separate from the feces in the rear section, thereby preventing undesirable mixing of the two bodily wastes. As such, the improved diaper-like undergarment prevents movement of the feces into the person's vaginal or scrotal sack area, thereby reducing the likelihood of the person contracting UTIs, rashes or other diseases. Although present invention is suitable for both children and adults, in a preferred configuration the improved diaper or diaper-like undergarment is sized and configured to be worn by an adult. The separation device of the present invention can be incorporated within or attached to a diaper or diaper-like undergarment to provide a barrier which substantially prevents movement of feces from the rear section to the front section of the undergarment and the movement of urine from the front section to the rear section of the undergarment, thereby preventing mixing of the bodily wastes and the UTIs, rashes or other infections, diseases or health problems that could otherwise result from the migration of such wastes.

In one embodiment, the present invention is an improved diaper or diaper like undergarment that is particularly and beneficially configured for persons suffering from dual incontinence. The undergarment has an outer diaper sheet, an inner layer and a separation device disposed at a middle area of the inner layer and configured to divide the inner layer into a front section at a front side of the undergarment and a back section at a back side of the undergarment. The separation device has a substantially liquid impenetrable upwardly projecting center divider that is configured to maintain urine in the front section of the inner layer and feces, and its associated liquids, in the back section of the inner layer. The center divider prevents mixing of urine and feces when the undergarment is being worn by a wearer suffering from dual incontinence. The center divider of the separation device interconnects the middle area of the undergarment with a centerpoint, defined as a position between the anus and the vagina/scrotal sack, of the wearer. Preferably, the improved undergarment includes a positioning means that is configured to maintain the proper positioning of the center divider at the centerpoint so as to maintain the required barrier to urine and feces migration. In a preferred embodiment, the positioning means comprises the upper surface of the center divider having an adhesive that removably attaches to the centerpoint of the wearer. For a pant style of undergarment, which has opposing side walls, the undergarment also has a side barrier on each side wall that interconnects the side wall with the wearer's body, typically at the hips, to prevent any migration of urine and/or feces along the side wall of the undergarment. In a preferred embodiment of the improved undergarment, the separation device has a first wing member extending outwardly from a first end of the center divider and a second wing member extending outwardly from a second end of the center section. The first and second wing members are configured to bend from an outward position to a downward position for placement between the legs of the wearer when the center divider is placed against the centerpoint. In the preferred embodiment, each of the first wing member and second wing member are configured to flex outward against the wearer's leg to assist in holding the center divider in place. To further assist in holding the center divider in place, each of the first wing member and the second wing member has an adhesive on their upper surfaces to removably attach to a leg of the wearer. In a preferred configuration, the middle area of the inner layer has a liquid impenetrable garment barrier that prevents migration of urine and/or feces between the front and back sections of the inner layer through the middle area of the undergarment. The undergarment can have a pair of marker lines at the middle area to identify where the wearer, or someone on his or her behalf, should position the center divider of the separation device.

In another embodiment, the present invention is a separation device that is utilized with existing diaper or diaper like undergarments to retrofit an undergarment and separate its inner layer into a front section and a back section in order to maintain urine in the front section and feces in the back section in a manner that prevents mixing of urine and feces when the undergarment is worn by a wearer. The preferred separation device comprises a substantially liquid impenetrable upwardly projecting center divider, a first wing member extending outwardly from a first end of the center divider and a second wing member extending outwardly from a second end of the center divider. As with the above embodiment, the center divider is sized and configured to be placed at a middle area of the inner layer so as to interconnect the middle area with a centerpoint of the wearer and provide a barrier preventing any migration of urine and feces. In a preferred configuration, each of the first wing member and the second wing member are configured to bend from an outward position to a downward position to facilitate placement of the center divider at the centerpoint of the wearer and to flex outward to press against the legs of the wearer in order to assist in holding the center divider in place. Each of the first wing member and the second wing member can have an adhesive on the upper or leg facing surface that is selected so as to removably attach the wing members to the legs of the wearer. If desired, this separation device can be included as part of a kit having a container of spray adhesive that is utilized to create a garment barrier at the middle area of the inner layer, a barrier sheet having a plurality of side barriers to prevent migration of feces along the side walls of the undergarment and an instruction booklet that describes how to use the separation device, spray adhesive and side barriers to prevent migration and mixing of urine and feces in an undergarment worn by a person suffering from dual incontinence.

The present invention also discloses the positioning means being a harness device that can be worn by a person wearing an improved undergarment or a separation device in an existing undergarment. In one embodiment, the harness is external to the undergarment. In another embodiment, the harness is incorporated into the undergarment. In either configuration, the harness is configured to apply and maintain pressure against the underside of the middle area of the undergarment so as to press the center divider of the separation device against the centerpoint of the wearer. In a preferred embodiment, the harness comprises an adjustable harness waist band worn about the wearer's waist and a pair of adjustable cross bands that define a pair of leg openings that receive the wearer's legs and a pressure point that presses against the bottom of the separation device to hold the center divider in place.

Accordingly, one of the primary aspects of the present invention is to provide an improved diaper or diaper-like undergarment and separation device for undergarments that has the advantages discussed above and which overcomes the disadvantages and limitations associated with presently available diaper and diaper-like products.

It is an important aspect of the present invention to provide an improved diaper or diaper-like undergarment that includes a barrier or separation device that separates the inner layer of the undergarment into front and back sections in a manner that prevents migration of urine and feces between the two sections and, therefore, mixing of the urine and feces.

It is also an important aspect of the present invention to provide an improved diaper or diaper-like undergarment that has a barrier or separation device that separates the front and back sections of the undergarment in a manner that prevents or at least substantially reduces the likelihood of the mixing of urine and feces and the migration of feces into the vaginal or scrotum area so as to reduce the likelihood of UTIs, rashes or other infections and/or health problems for the wearer of the undergarment and others who come into contact with such persons.

It is also an important aspect of the present invention to provide an improved diaper or diaper-like undergarment that reduces the indignity and health problems resulting from a person sitting in a mixture of urine and feces for an extended period of time.

It is also an important aspect of the present invention to provide a separation device that is configured to be made integral with or utilized with a diaper or diaper-like undergarment to provide a barrier that separates the front and back sections of the undergarment to prevent mixing of urine and feces and the migration of feces into the vaginal or scrotum area so as to prevent or reduce the likelihood of UTI and other infections, diseases and/or health problems.

Yet another important aspect of the present invention is to provide a separation device for diaper and diaper-like undergarments that can be utilized as part of a kit which allows conversion of a standard undergarment to one having a barrier between the front and back sections of the undergarment that prevents mixing of urine and feces and migration of feces into the vaginal or scrotum area of the wearer's body.

Yet another important aspect of the present invention is to provide an improved diaper or diaper-like undergarment and separation device for such undergarments that is sized and configured for use by adults.

The above and other aspects and advantages of the present invention are explained in greater detail by reference to the attached figures and the description of the preferred embodiment which follows. As set forth herein, the present invention resides in the novel features of form, construction, mode of operation and combination of the above presently described and understood by the claims.

### BRIEF DESCRIPTION OF THE DRAWINGS

In the drawings which illustrate the preferred embodiments and the best modes presently contemplated for carrying out the present invention:

FIG. 1 is a top view of a first or diaper style undergarment configured according to a first embodiment of the present invention;

FIG. 2 is a top view of a second or single unit version (pants style) undergarment configured according to a second embodiment of the present invention;

FIG. 3 is a front view of the pant style undergarment of FIG. 2 shown on a wearer and utilizing a specially configured separation device having the wing members in the downward position;

FIG. 4 is a front view of the separation device of FIG. 3 showing the separation device in both its outward position and its downward position, with the arrow indicating the movement thereof, shown positioned over the middle area of an undergarment;

FIG. 5 is a top view of the separation device of FIG. 4 showing a cover over the upper surfaces of the center divider and wing members;

FIG. 6 is a top view of the separation device of FIG. 5 showing the cover removed to expose the adhesive on the upper surfaces;

FIG. 7 is a bottom view of the separation device of FIG. 6 showing the cover over the lower surface of the center divider;

FIG. 8 is a bottom view of the separation device of FIG. 7 showing the cover removed to expose the adhesive layer on the lower surface of the center divider;

FIG. 9 is a side perspective view of the center divider of FIG. 4;

FIG. 10 is a cross sectional side view of the separation device and middle area of the undergarment taken through lines 10-10 of FIG. 4;

FIG. 11 is a top view of the middle area of the undergarment of FIG. 10 showing the marking lines thereon for positioning the center divider of the separation device;

FIG. 12 shows the various components of a kit utilizing the separation device of FIG. 4 to retrofit an existing undergarment;

FIG. 13 is a side perspective view of a harness that can be utilized to apply additional pressure against the separation device of the present invention; and

FIG. 14 is a top view of the harness of FIG. 13.

### DETAILED DESCRIPTION OF THE PREFERRED EMBODIMENTS

With reference to the figures where like elements have been given like numerical designations to facilitate the reader's understanding of the present invention, the preferred embodiments of the present invention are set forth below. The enclosed text and drawings are merely illustrative of one or more preferred embodiments and, as such, disclose one or more different ways of configuring the present invention. Although specific components, materials, configurations and uses are illustrated, it should be understood that a number of variations to the components and to the configuration of those components described herein and in the accompanying figures can be made without changing the scope and function of the invention set forth herein. For instance, although the figures and description provided herein show certain shapes and configurations for a diaper or diaper-like undergarment, those skilled in the art will readily understand that this is merely for purposes of simplifying the present disclosure and that the present invention is not so limited.

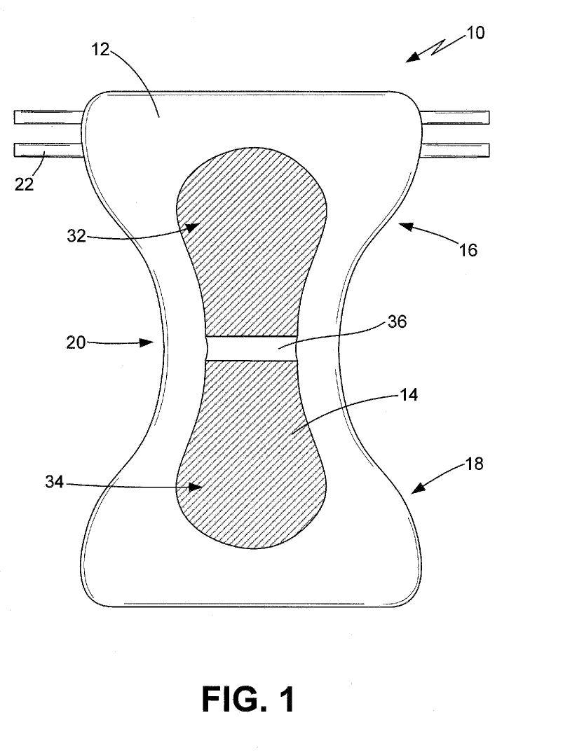

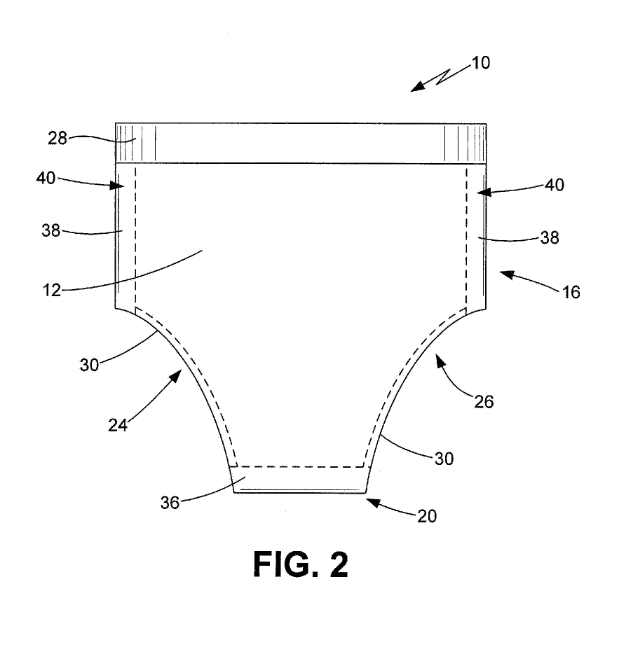

An improved diaper or diaper-like undergarment that is configured pursuant to one embodiment of the present invention is shown generally as 10 in FIGS. 1 and 2. As with presently configured diaper or diaper like undergarments, the undergarment 10 of the present invention has a layered construction that comprises an outer diaper sheet 12, which is typically made of liquid impenetrable shell material that is chosen to generally prevent migration of bodily wastes outside of the undergarment 10, and an inner layer 14 that lays next to the skin and which is made out of materials that are chosen for their ability to transfer wetness away from the body and to absorb urine and other liquids. Although not shown in the drawings, those skilled in the art readily understand that the inner layer 14 typically comprises two separate functional layers, a contact layer that lays next to the body and an absorbent layer which is disposed between the contact layer and the outer diaper sheet 12. The material for the contact layer is chosen for its softness and its ability to transfer liquids to the absorbent layer. The absorbent layer is chosen for its ability to absorb liquids and retain those liquids away from the body. As best shown in FIG. 1, the inner layer 14 generally extends from the front side 16 of the undergarment 10 to the back side 18 of the undergarment 10. As well known in the art, front side 16 of the undergarment 10 is configured to be placed against the front waist area of the wearer and back side 18 of the undergarment is configured to be placed against the back waist area of the wearer. The middle area 20 of the undergarment 10 is positioned under the crotch area of the wearer when the undergarment is secured on the wearer. The standard diaper configuration, shown in FIG. 1, utilizes securing tabs 22 to secure the top of the undergarment 10 around the wearer's waist area. Typically, the edges of the outer diaper sheet 12 at and near the middle area 20 of the undergarment 10 of FIG. 1 are configured in a manner that will reduce the likelihood human waste will exit the undergarment 10 when being worn by the wearer thereof. These configurations are well known in the art.

FIG. 2 illustrates a different style of undergarment 10. This style is commonly referred to as a pant-type of undergarment because it is put on like a pair of pants, with one leg placed through a first leg opening 24 and the other leg placed through the second leg opening 26. This style of undergarment typically includes an elastic waist band 28 that secures around the user's waist. The leg openings 24 and 26 are provided with an elastic leg band 30 that secures around the user's leg to prevent migration of bodily wastes out of the undergarment 10 around the leg.

The inner layer 14 of prior art undergarments have a front section 32 that is continuous with a back section 34. Unfortunately, this configuration results in the urine and feces being able to migrate between the front 32 and back 34 sections and mix, creating an unhealthy mixture of urine and feces that lays against the wearer's skin. As discussed above, the feces or mixture of urine and feces can cause a UTI in females and rashes along the front, back and crotch areas of both females and males, which can lead to serious infections, diseases and other medical conditions. Treating such infections and diseases can result in overuse of antibiotics, which can lead to antibiotic resistant diseases.

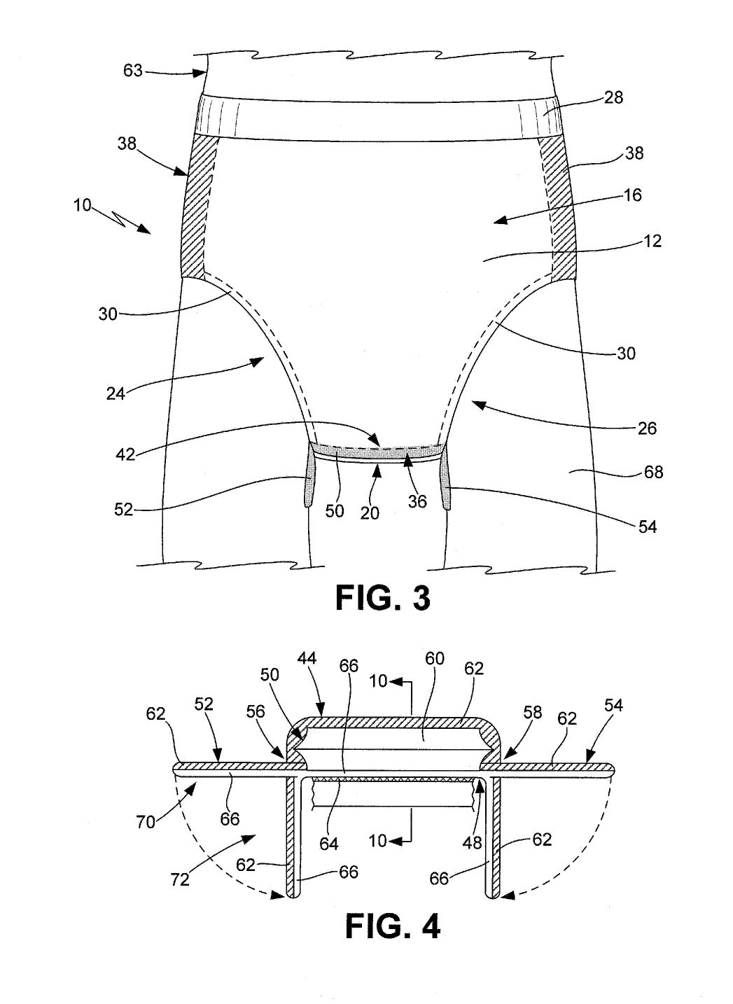

As discussed in more detail below, the improved undergarment 10 of the present invention includes a separation device 36 in the middle area 20 of the undergarment 10 that separates the front section 32 and back section 34 from each other so as to prevent the migration of urine from the front section 32 to the back section 34 and the migration of feces from the back section 34 to the front section 32. The improved pant style undergarment 10 of the present invention, shown in FIG. 2, also includes side barrier 38 on each of the opposing side walls 40 of the undergarment 10. The side barriers 38 are configured and positioned to make sure there is no migration of urine or feces between the front 32 and back 34 sections along the side walls 40 of undergarment 10. The separation device 36 is disposed in middle section 20 such that it will be appropriately positioned in the wearer's crotch area when he or she is wearing the undergarment 10 of the present invention. The side barriers 38 are generally aligned with the separation device 36. The separation device 36 will fit snugly and comfortably against the skin of the user's crotch area to press firmly, yet gently, against a point between the anus and vagina/scrotal sack, hereinafter referred to as the centerpoint 42, as shown in FIG. 3. Although a wide variety of different materials can be utilized for undergarment 10, with such materials being well known in the art, in the preferred embodiment both the diaper and pant style undergarment 10 are made out of disposable materials.

The separation device 36 must be made from or coated/covered with a liquid impenetrable material that will prevent the migration of urine from the front section 32 to the back section 34 and the migration of feces from the back section 34 to the front section 32. In a preferred embodiment, the separation device 36 comprises a plastic or plastic like material that is non-soluble. The separation device 36 utilized with an embodiment of the present invention having an undergarment 10 that is intended to be washable and reusable, must be made out of materials that are selected to withstand repeated washing and drying or be configured to be replaceable between uses.

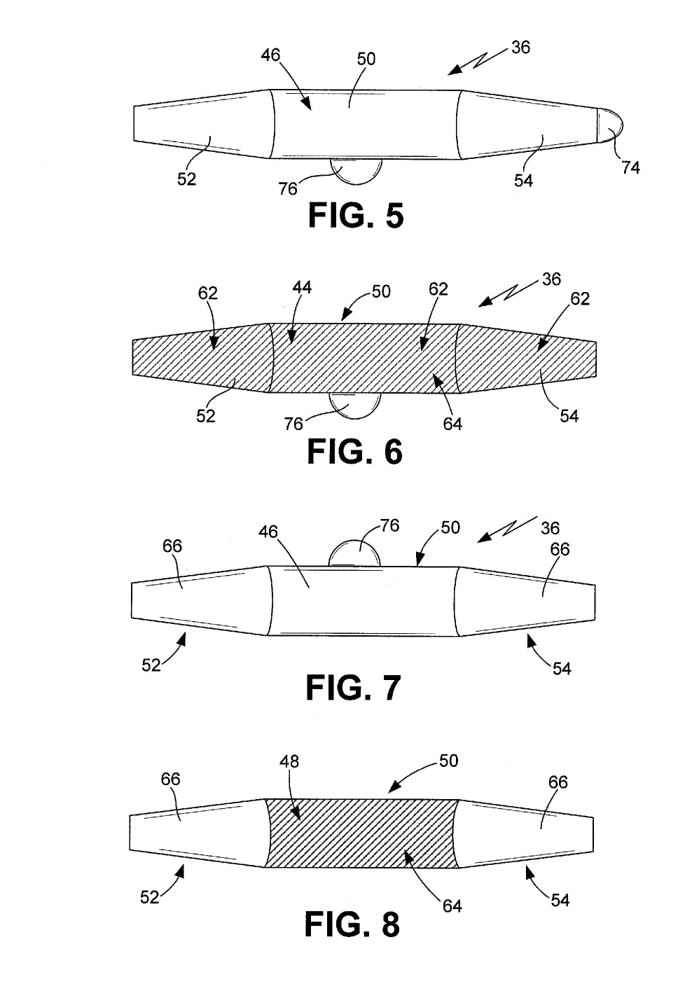

To assist with holding the separation device 36 in place, the present invention also includes a positioning means that is configured to maintain the proper positioning of the separation device 36 relative to the centerpoint 42 on the wearer's body. In a preferred embodiment, upper surface 44 of separation device 36 is coated, impregnated or otherwise provided with a non-water soluble, non-allergenic adhesive that has an adhesive quality generally similar to that utilized with commonly available bandages. A peel off cover 46, described in conjunction with the embodiments of FIGS. 5 through 8, will be over the upper surface 44 until the undergarment 10 is ready to be used. After removal of the peel off cover 46, the sticky upper surface 44 is placed against the centerpoint 42. The tabs 22 utilized with the diaper style undergarment 10 and the elastic waist band 28 utilized with the pant style undergarment 10 should be configured to tightly secure the undergarment 10 to the wearer so the middle area 20 of undergarment 10 will help hold the separation device 36 in place and maintain effective separation of the front 32 and back 34 sections. As well known in the art, a wide variety of different types of tabs 22 and waist bands 28 can be utilized with the undergarment 10 of the present invention, including tape, Velcro®, snaps, ties or other fastening devices. The fastening device should be configured so that it is easy for the wearer to use himself or herself or for persons at the hospital or other health care facility to use. If desired, the undergarment 10 can be configured with a tape-hatched hole large enough for catheters to be used in conjunction with the undergarment 10.

The outer shell or diaper sheet 12 of the undergarment 10 will be made of biodegradable plastic material sufficient to keep the outer clothing and bedding materials with which the wearer of the undergarment 10 comes into contact from becoming wet or soiled due to such contact. As set forth above, the material paper and cloth of inner layer 14 will be layered in a manner that draws moisture away from the body. In a preferred embodiment, undergarment 10 will also contain a pleasant smelling powder or non-scented optional powder toward the outer layers, most distal from the body, so the when the powder comes into contact with moisture (i.e., urine or loose stools), the moisture will thicken at or near the plastic outer shell 12. Once the moisture is thickened it will be less likely to return to make contact with the skin of the individual wearing it. Fixing it in place in this fashion with the scent neutralizing agent will also result in less odor problems. If desired, the back section 34 can have optional inserts of multiple heat sensitive non allergenic thin biodegradable plastic-like inserts, which upon contact with warm formed stools, will automatically cause them to wrap around the stool(s), further keeping them away from the individuals skin and further reducing any foul odor. This will be accomplished using thin memory plastic sheeting that has been flattened and separated by paper padding on both sides. The memory will roll back into the shape of the stool to keep the stool away from the body.

Not only does the undergarment 10 of the present invention serve the health interests of the world population by preventing UTIs and various other diseases known to be caused by the avoidable over-usage of antibiotics, as set forth above, but reducing the mixture of urine and feces, keeping the individual dry and more odor free, will restore a greater degree of personal dignity to older people and others who are dual incontinent. In addition, the present invention allows persons with incontinence problems to have greater mobility in society, without the embarrassment of bad smell and being required to sit in direct contact with their own mixed, moist excrement and urine. The undergarment 10 of the present invention can be provided in fitted models for both the diaper and pant style undergarments 10, thereby providing a lower visible profile to lessen the appearance of wearing such undergarment 10.

Another benefit of the undergarment 10 of the present invention is that it will allow medical professionals to spend more time treating their patients, rather than spending so much time trying to clean them after they have soiled themselves. These additional hours of available health care can be used to provide beneficial treatment instead of hygiene maintenance, thus allowing for more and better healthcare in general. As well known in the medical field, one of the reasons that medical workers in contact with senior citizens and other who are incontinent become depressed and discouraged is that they see their roll in life as being the person who spends a major part of their job wiping and cleaning the genitals and anuses of others. This invention makes the entire process less messy and easier to accomplish. As a result, the healthcare profession will be less depressed and pass this more positive state of mind on to the patient, as opposed to making the patient feel like a burden.

Although the discussion herein is primarily directed to embodiments for adults, the undergarment 10 of the present invention can be manufactured in sizes suitable for children, infants and adults.

In use, the individual who will wear undergarment 10 or his or her caregiver determines the appropriate or best fit adhesive separation device 36 for the wearer's body at the centerpoint 42 and the best fit side barriers 38 for the side walls 40 (for the pant style undergarment 10). The size of the undergarment 10 itself should be selected so as to snugly fit the wearer. After removing the covers 46 from the lower 48 (best shown in FIG. 8) and upper surface 44 of the separation device 36, the wearer or his or her caregiver sticks the lower side 48 of the separation device 36 to the middle area 20 of the undergarment 10, which should be made out of plastic or other liquid impenetrable material to prevent migration of liquid around the separation device 36 through the undergarment, and then sticks the sticky upper surface 44 against the user's skin at the centerpoint 42. The undergarment 10, whether the diaper style or the pant style, is then put on as it would normally be put on and snugly attached to the wearer's body to achieve the best separation of the two sections 32/34. For the pant style of undergarment 10, any covers on the side barriers 38 located at side walls 40 are removed and the side barriers 38 are placed against the wearer (i.e., at or near the hips). To ensure separation of the two sections 32/34, the wearer or his or her caretaker should hand press the undergarment 10 at the middle area 20 and the side walls 40 to obtain full contact of the separation device 36 and the side barriers 38 with the wearer's body and be sure they are in place. After use, the undergarment 10 is removed from the wearer, taking all reasonable and/or required hygienic precautions, and thrown away by placing in a proper disposal bag in compliance with prevailing state and federal law for such products. The body area of the wearer is cleaned with moist and dry soft cloth or paper products and powdered for dryness and sanitation. The undergarments 10 is disposed of as required by law. Preferably, the person handling a used undergarment should always use rubber gloves. Typically, in a hospital or nursing home environment, the caregivers will use rubber gloves when applying and removing undergarment 10 from the wearer. If the undergarment 10 is washable and reusable, it is taken off of the wearer, handling as described above, the wastes are disposed of and the undergarment 10 washed and dried as is typical for such products. In some embodiments, the separation device 36 will be removed from the undergarment 10 and disposed of after use and then replaced when placing a clean undergarment 10 on the wearer.

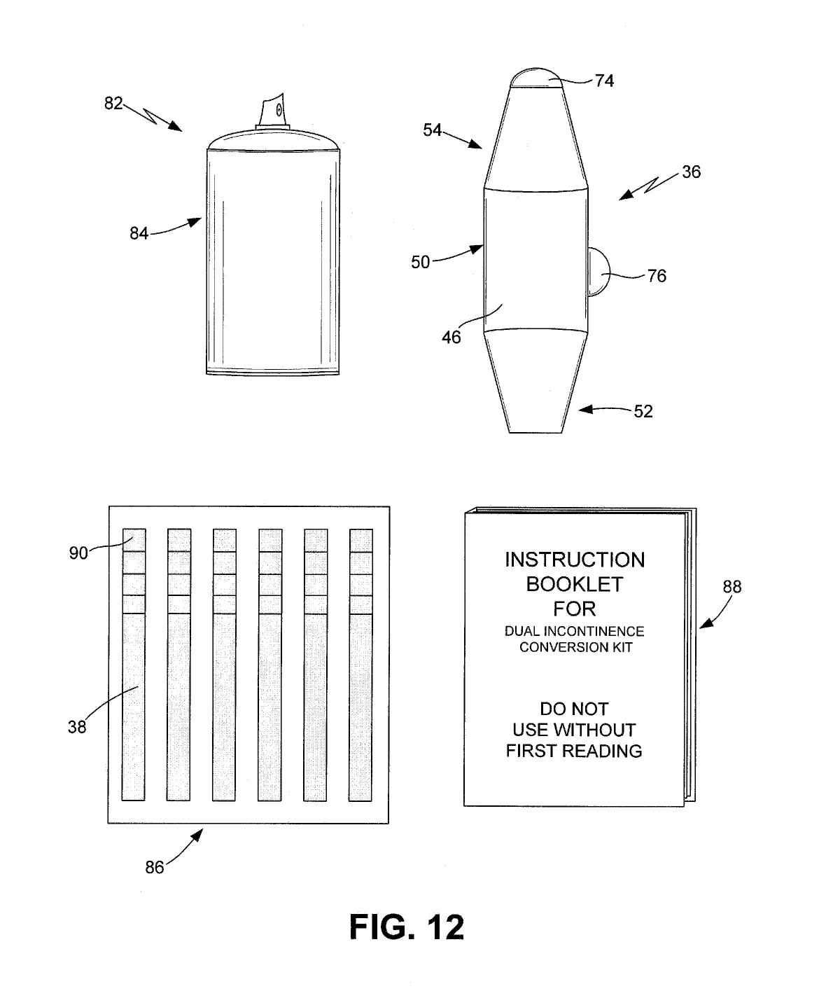

A preferred embodiment of the separation device 36 of the present invention is shown in FIGS. 3 through 11 and a preferred kit for use with this separation device 36 is shown in FIG. 12. Although these figures generally show use of the separation device 36 with a pant style undergarment 10, the separation device 36 is not so limited. The separation device 36 shown in FIGS. 3 through 11 and described below can be utilized with the diaper style undergarment 10 and with other styles of undergarments 10 that are presently available or which may become available in the future. The separation device 36 of the preferred embodiment generally comprises a center divider 50, first wing member 52 and second wing member 54. Center divider 50, which in a preferred embodiment is elongated and has a first end 56 and a second end 58, is sized and configured to be placed at the middle area 20 of the undergarment 10 and against centerpoint 42 to block any migration of urine from the front section 32 to the back section 34 and migration of feces from the back section 34 to the front section 32, as well as any mixing of the two bodily wastes. As best shown in FIG. 4, the center divider 50 projects above the plane through the wing members 52/54. The first wing member 52 extends outwardly from the first end 56 of the center divider 50 and the second wing member 54 extends outwardly from the second end 58 of center divider 50.

As best shown in FIG. 4, the preferred embodiment of center divider 50 comprises a center core 60 that is configured to be liquid impenetrable so as to provide the migration blocking benefits of center divider 50 described above. In one configuration, the center core 60 is made of a sponge-like material that is impregnated or coated with a resin, polymer or like material to form a flexible yet liquid impenetrable barrier. In the preferred embodiment, wherein the positioning means comprises the use of an adhesive on the separation device 36 that sticks to the centerpoint 42 of the wearer 63 (shown in FIG. 3), the center core 60 is covered with a contact layer 62 that is made out of a material or materials that will be comfortable against the wearer 63 of undergarment 10, such as soft cloth or paper material. The body contact layer 62 defines the upper surface 44 of the separation device 36 and is coated or impregnated with an inert, non-allergenic adhesive, shown as 64 in FIG. 6, that is suitable for holding separation device 36 against the wearer 63 at centerpoint 42. As described above, the preferred adhesive 64 is the type utilized for bandages and the like, which stick well against human skin but are relatively easily and painlessly removed. The bottom surface 46 of separation device 36 is defined by a relatively thin, bendable outer layer 66 that is selected so as to allow the wings 52/54 to bend generally downward, as shown in FIG. 4. As shown in FIG. 3, when separation device 36 is in use, the adhesive 64 on contact layer 62 contacts the wearer's skin, both at centerpoint 42 and at the inside of his or her legs 68 and outer layer 66 is disposed outwardly from the wearer's skin. In the preferred embodiment, the material for outer layer 64 is nylon or a like material that can be elastically deformed so that when wings 52/54 are bent downward, as shown in FIGS. 3 and 4, they will apply pressure against the inside of the wearer's legs 68 to assist in holding the separation device 36 in place against the centerpoint 42. The use of the bendable material that will flex outward against the wearer's legs 68 is a further positioning means, which can be utilized with or without the adhesive, that assists in holding the center divider 50 in its proper position against the centerpoint 42 of the wearer 63 to function as a barrier which prevents migration of urine and feces.

The center divider 50 will be sized for different sizes of wearers of undergarment 10. In one embodiment, an undergarment 10 can be supplied with a plurality of different sizes of center dividers 50 so the wearer 63 can choose the size that best fits him or her. In another embodiment, the wearer 63 can be supplied with a sizing chart that he or she, or someone on his or her behalf, can determine the best size center divider 50 and purchase or order the separation device 36 having that size center divider 50. If desired, the portion of the outer layer 66 extending down the legs 68 of the wearer 63 can be covered with a glossy, smooth plastic layer to keep the nylon outer layer 66 from sticking to the wearer's clothing.

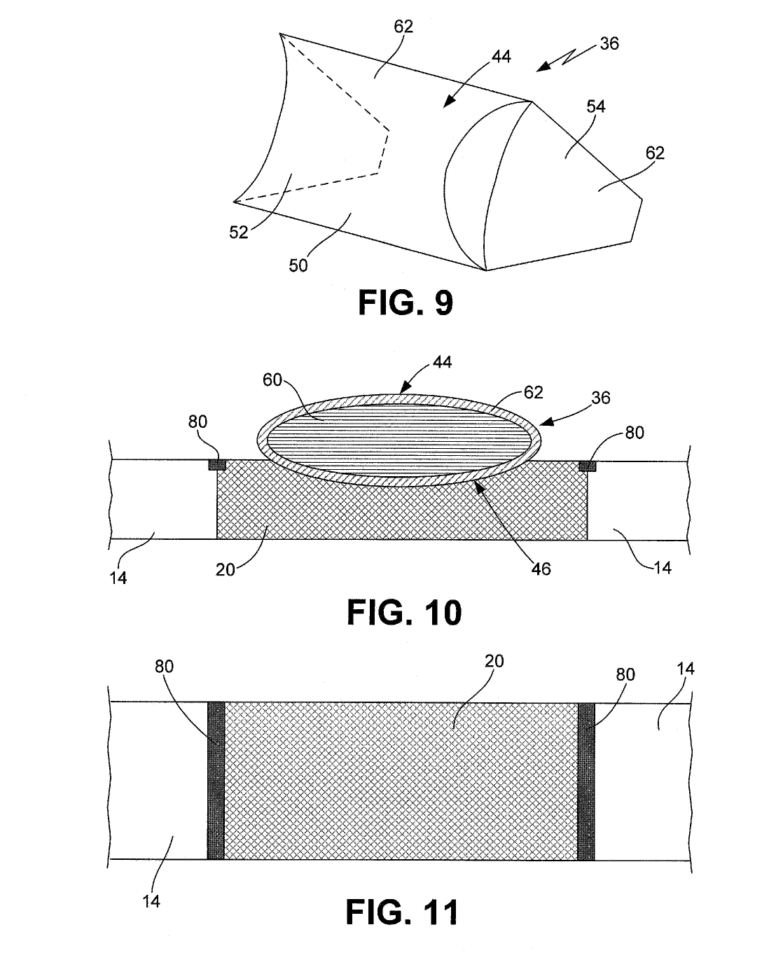

As set forth above and shown in FIGS. 4 through 8, the first 52 and second 54 wing members extend outwardly from the first 56 and second 58 ends, respectively, of the center divider 50 and are configured to move from a straight outward position 70 to a generally downward position 72, as shown in FIGS. 4 and 9. In the preferred embodiment, the materials for the contact layer 62 and the outer layer 66 extend contiguously across the first wing member 52, center divider 50 and second wing member 54, as shown in FIG. 4. Likewise, the adhesive layer 64 extend across these components, as shown in FIG. 6, to contact the wearer's skin at the centerpoint 42 and against his or her legs 68. In this embodiment, the positioning means further comprises the adhesive being extended to the wing members 52/54 such that he adhesive 64 on upper surface 44 and contact layer 62 will attach the separation device 36 to the centerpoint 42 and to the inside of the user's legs, as shown in FIG. 3. The adhesive 64 on the lower surface 48 will attach the center divider 50 portion of the separation device 36 to the middle area 20 of the undergarment 10.

In use, the wearer 63 of undergarment 10 or a caretaker on his or her behalf will size the center divider 50 portion of the separation device 36 for user at centerpoint 42. Typically, separation device 36 will be provided with the wing members 52/54 in their outward position 70 with a peel off cover 46 extending across and protecting the adhesive 64 on the upper surface 44 of the center divider 50 and across the first 52 and second 54 wing members, as shown in FIG. 5. In a preferred embodiment, an end pull tab 74 is connected to the cover 46 over these components to facilitate removal of the cover 46 and exposure of the adhesive 64, as shown in FIG. 6. The wearer 63 or caretaker will then bend back wing members 52/54 to the downward position shown in FIGS. 3, 4 and 9, and place the center divider 50 against the centerpoint 42 of the wearer 63 and against the inside of his or her legs 68, adhering to both the centerpoint 42 and legs 68. When the wearer 63 or caretaker lets go of the wings 52/54, they will attempt to straighten out and, in doing so, press against the inside of legs 68 to help assist holding separation device 36 in place. The wearer 63 or his or her caretaker then peels off the cover 46 over the lower surface 48 by, in a preferred configuration, pulling a center tab 76, shown in FIGS. 5 through 7, to expose the adhesive 64 on the lower surface 48 of center divider 50, as shown in FIG. 8. The separation device 36 will be in the position shown in FIGS. 3, 4 and 9. The wearer 63 or his or her caretaker then places the middle area 20 of undergarment 10 on the wearer 63, preferably positioning the center divider 50 as set forth below, and then puts the rest of the undergarment 10 on the wearer 63, as is shown in FIG. 3 with regard to the pant style undergarment 10. In the preferred embodiment, the waist band 28 is snugly fit around the wearer's waist to assist in holding the separation device 36 in place against centerpoint 42 and middle area 20 to provide the desired barrier to any migration and mixture of urine and feces across the middle area 20 of the undergarment 10. To prevent any migration of urine and/or feces across the side walls 40 of the pant style undergarment 10, the undergarment 10 should have or the wearer/caretaker should install one or more side barriers 38 on each of the opposing side walls 40 of undergarment 10. As described above, the side barriers 38 will also stick to the hips of the wearer 63 of undergarment 10 to ensure that no urine or feces will pass along side walls 40.

In the preferred embodiment of the undergarment 10 for use with the separation device 36 of the present invention, the undergarment is modified to facilitate use with separation device 36 and the effectiveness thereof. One such modification is to provide a liquid impenetrable garment barrier 78 at the middle area 20 of undergarment 10 across the entire width or substantially the entire width of undergarment 10 at middle area 20, as shown in FIGS. 10 and 11. In a preferred embodiment, middle area 20 comprises an impermeable polymer or resin material that will not let any urine or feces pass through between the front 32 and back 34 sections. To facilitate the correct placement of center divider 50 of the separation device 36, the undergarment 10 can include a pair of marker lines 80, as shown in FIGS. 10 and 11, that define the outer boundaries of the garment layer 78. In one embodiment, the marker lines 80 are red or some other contrasting color so they may be easily identified as the location where the center divider 50 is to be positioned. Likewise, the end tab 74 and the center tab 76 can also be provided with contrasting colors to help facilitate proper removal of the cover 46 over the adhesive 64 on upper surface 44 and lower surface 48. In the preferred embodiment, the outer diaper sheet 12 is made stronger than present undergarments to better facilitate the wearer 63 or his or her caretaker snugly pulling the undergarment 10 on to better hold the separation device 36 in place against centerpoint 42 and middle area 20. Likewise, preferably the waist band 28 and or any securing tabs 22 are made stronger and more secure in order to better hold the undergarment 10 in place around the wearer's waist and, therefore, better hold the separating device 36 in place. In one embodiment, an inner lining of cloth or like material can be placed at waist band 28 to facilitate snug fitting of the undergarment 10 around the wearer's waist.

In one embodiment of the present invention, the separation device 36 can be provided as part of kit, shown as 82 in FIG. 12, for retrofitting existing undergarments with the benefits of the present invention, namely separation of the front 32 and back 34 sections of the inner layer 14 so as to prevent urine and/or feces from migrating between sections 32/34 and mixing together to form a mixture that is unhealthy for the wearer 63 of the undergarment. In a preferred embodiment, the kit 82 comprises separation device 36, a can or other container 84 of non-allergenic adhesive material, a barrier sheet 86 having a plurality of side barriers 38 thereon and an instruction booklet 88 that provides instructions on how to use the kit 82 to convert an existing undergarment for use with persons having dual incontinence problems. The container 84 should contain adhesive material that is configured to penetrate the middle area 20 of the undergarment and form a garment barrier 78 sufficient to substantially prevent any migration of urine and/or feces. In a preferred embodiment, container 84 is a spray container. The barrier sheet 86 should contain side barriers 38 having barrier segments 90 that can be removed from the side barriers 38 as is necessary to provide an appropriately sized side barrier 38 for the side walls 40 of the undergarment. If desired, the instruction booklet 88 can include a warning that the use of specially made undergarments 10 is preferred due to the possible inaccuracy of the wearer 63 or his or her caretaker forming undergarment 10. The separation device 36 and side barriers 38 are utilized as described above. If desired, the kit 82 can contain different sizes of center divider 50 to allow the wearer 63 or his or her caretaker to select the best fitting size.

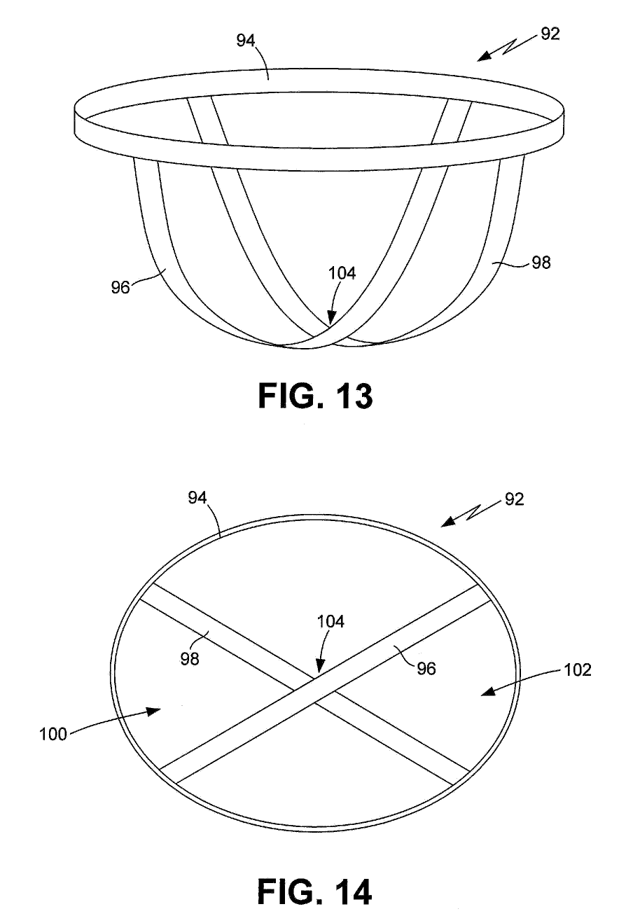

FIGS. 13 and 14 show a harness 92 that can be worn by the wearer 63 of undergarment 10 or an undergarment retrofitted with the kit 82 to apply pressure to the separation device 36 to better maintain the barrier between the front section 32 and the rear section 34 so as to prevent migration of urine and feces, as described above. The harness 92 can be utilized alone as the positioning means or it can be utilized with the adhesive 64 and/or the flexing wings 52/54 described above. Preferably, the harness 92 has an adjustable harness waist band 94 that is configured to go around the wearer's waist. In a preferred embodiment, the harness waist band 94 has a clingy rubber-like inside component that better secures around the wearer's waist. As shown in FIGS. 13 and 14, the preferred harness 92 also comprises a pair of elastic cross bands 96 and 98, which are preferably configured to be adjustable, disposed in a generally X-shaped configuration to form a right leg opening 100, a left leg opening 102 and a device pressure point 104. The right leg of the wearer 63 is inserted into the right leg opening 100 and the left leg of the wearer 63 is inserted into the left leg opening 102. Alternatively, the harness waist band 94 and cross bands 96/98 are configured to be separated for placement under or around the wearer 63, which is particularly useful for the bedridden wearer 63. The harness 92 is then pulled up so the device pressure point 104 is pressed against the underside of the middle area 20 of the undergarment 10 to press the center divider 50 of the separation device 36 against the centerpoint 42 to better provide a barrier against migration of urine and feces. As readily apparent to those skilled in the art, a variety of different configurations of harness 92, instead of the X-shaped configuration shown in FIGS. 13 and 14, can be utilized to assist in maintaining the positioning of the center divider 50 against centerpoint 42. The harness 92 can be a separate external device which is placed around the outside of the undergarment 10 or it can be incorporated into the undergarment 10, either on the outside thereof or inside the undergarment 10.

While there are shown and described herein specific embodiments of the present invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to any dimensional relationships set forth herein and modifications in assembly, materials, size, shape and use. For instance, there are numerous components described herein that can be replaced with equivalent functioning components to accomplish the objectives of the present invention.

What is claimed is:

1. An undergarment, comprising:
an outer diaper sheet;
an inner layer; and
a separation device disposed at a middle area of said inner layer and configured to divide said inner layer into a front section at a front side of said undergarment and a back section at a back side of said undergarment, said separation device having a liquid impenetrable upwardly projecting center divider configured to maintain urine in said front section and feces in said back section so as to prevent mixing of urine and feces when said undergarment is worn by a wearer, said separation device substantially interconnecting said middle area of said undergarment with a centerpoint of the wearer.

2. The undergarment of claim 1, wherein said undergarment further comprises a pair of opposing side walls and a side barrier disposed on each of said side walls generally aligned with said separation device, each of said side barriers configured to attach to the wearer so as to interconnect said side walls of said undergarment with said wearer to prevent migration of urine and feces along said side walls.

3. The undergarment of claim 1 further comprising positioning means associated with said undergarment for positioning said center divider of said separation device generally against the centerpoint to prevent migration and mixing of urine and feces.

4. The undergarment of claim 3, wherein said positioning means comprises an adhesive on said upper surface of said center divider, said adhesive selected to attach said center divider to said centerpoint.

5. The undergarment of claim 3, wherein said positioning means comprises a first wing member extending outwardly from a first end of said center divider and a second wing member extending outwardly from a second end of said center section when said first wing member and said second wing member are in an outward position, each of said first wing member and said second wing member configured to bend between said outward position and a downward position and flex outwardly to press against a leg of the wearer.

6. The undergarment of claim 5, wherein each of said first wing member and said second wing member has an adhesive thereon configured to attach to the leg of the wearer.

7. The undergarment of claim 6, wherein said positioning means further comprises an adhesive on said upper surface of said center divider, said adhesive selected to attach said center divider to the centerpoint.

8. The undergarment of claim 3, wherein said positioning means comprises a harness configured to apply pressure to said separation device to press said center divider generally against the centerpoint.

9. The undergarment of claim 1, wherein said middle area of said inner layer has a liquid impenetrable garment barrier to prevent migration of urine and/or feces between said front section and said back section through said middle area of said undergarment.

10. An undergarment, comprising:
a liquid impenetrable outer diaper sheet;
an inner layer configured to absorb liquids, said inner layer having a middle area with a liquid impenetrable garment barrier configured to prevent migration of urine and/or feces through said middle area;
a separation device disposed at a middle area of said inner layer and configured to divide said inner layer into a front section at a front side of said undergarment and a back section at a back side of said undergarment, said separation device having a liquid impenetrable upwardly projecting center divider configured to maintain urine in said front section and feces in said back section so as to prevent migration and mixing of urine and feces when said undergarment is worn by a wearer; and
positioning means associated with said undergarment for positioning said center divider of said separation device generally against the centerpoint to prevent migration and mixing of urine and feces.

11. The undergarment of claim 10, wherein said positioning means comprises an adhesive on said upper surface of said center divider, said adhesive selected to attach said center divider to said centerpoint.

12. The undergarment of claim 10, wherein said positioning means comprises a first wing member extending outwardly from a first end of said center divider and a second wing member extending outwardly from a second end of said center section when said first wing member and said second wing member are in an outward position, each of said first wing member and said second wing member configured to bend between said outward position and a downward position and flex outwardly to press against a leg of the wearer.

13. The undergarment of claim 12, wherein each of said first wing member and said second wing member has an adhesive thereon configured to attach to the leg of the wearer.

14. The undergarment of claim 13, wherein said positioning means further comprises an adhesive on said upper surface of said center divider, said adhesive selected to attach said center divider to the centerpoint.

15. The undergarment of claim 14, wherein said positioning means further comprises a harness configured to apply pressure to said separation device to press said center divider generally against the centerpoint.

16. The undergarment of claim 10, wherein said positioning means comprises a harness configured to apply pressure to said separation device to press said center divider generally against the centerpoint.

17. The undergarment of claim 10, wherein said undergarment further comprises a pair of opposing side walls and a side barrier disposed on each of said side walls generally aligned with said separation device, each of said side barriers configured to interconnect said side walls of said undergarment with said wearer to prevent migration of urine and feces along said side walls.

18. A separation device for use with an undergarment worn by a wearer to separate an inner layer of said undergarment into a front section and a back section so as to maintain urine in said front section and feces in said back section and prevent mixing of urine and feces when said undergarment is worn by the wearer, said separation device comprising:
a substantially liquid impenetrable upwardly projecting center divider sized and configured to be placed at a middle area of said inner layer so as to interconnect said middle area with a centerpoint of the wearer;
a first wing member extending outwardly from a first end of said center divider; and
a second wing member extending outwardly from a second end of said center divider, each of said first wing member and said second wing member configured to bend from an outward position to a downward position to facilitate placement of said center divider at the centerpoint of the wearer.

19. The undergarment of claim 18, wherein each of said first wing member and said second wing member is configured to flex outwardly and press against a leg of the wearer.

20. The undergarment of claim 18, wherein each of said first wing member and said second wing member has an adhesive thereon configured to attach to the leg of the wearer.
